(19) United States
(12) Patent Application Publication
(10) Pub. No.: US 2023/0270379 A1
(43) Pub. Date: Aug. 31, 2023

(54) DEVICE FOR MEASUREMENT OF PHYSIOLOGICAL PARAMETERS THROUGH A SKIN CONTACT SURFACE

(71) Applicant: CARDIACSENSE LTD., Caesarea (IL)

(72) Inventors: Boris SPEKTOR, Haifa (IL); Andres WAINSTEIN, Ramat Gan (IL); Eldad SHEMESH, Binyamina (IL); Gustavo TURKIELTAUB, Tel Aviv (IL)

(21) Appl. No.: 18/006,952

(22) PCT Filed: Jul. 25, 2021
(86) PCT No.: PCT/IL2021/050898

§ 371 Date: Jan. 26, 2023

(30) Foreign Application Priority Data
Jul. 26, 2020 (IL) 276279

(51) Int. Cl. A61B 5/00; A61B 5/318

(52) U.S. Cl. CPC A61B 5/681; A61B 5/0059; A61B 5/318; A61B 5/6844; A61B 2562/06; A61B 2562/04; A61B 2562/12

(57) ABSTRACT

The present disclosure discloses a wearable device for measuring physiological parameters of a subject while being worn. The device includes one or more sensors that perform the measurement of the physiological parameters. The sensors can be selected from either a light-based sensor or displacement sensor. The device includes a skin contact member that has a skin contact surface facing an external side of the device that faces the skin of the subject. During measurements of one of the sensors, the contact surface engages the skin of the subject to allow performing measurements by at least one sensor. The light-based sensor is configured to perform the measurement via the skin contact surface, and the displacement sensor is configured to sense the displacement of the skin contact surface. For allowing accurate performance of the measurements, the skin contact surface is required to displace smoothly in response to contact or pressure by the skin of the subject thereon. Furthermore, the design of the skin contact surface should allow functional engagement with the skin of the subject. Thus, the skin contact member is integrally formed with a first end of a flexible member allowing the smooth displacement thereof. A housing of the device is integral with a second end of the flexible member such that the skin contact member displaces along at least one axis with respect to the static housing. The integration of the three elements, the skin contact member, the flexible member, and the housing forms a continuous structure, which is advantageous and allows to obtain the desired accuracy of the fine measurements. The integration of the three parts is typically performed by one or more over-molding processes.

### TECHNOLOGICAL FIELD

The present disclosure is in the field of medical measurement devices for measuring physiological parameters of a subject.

### BACKGROUND ART

References considered to be relevant as background to the presently disclosed subject matter are listed below:
US 2018/0146870
WO 2019/215723

Acknowledgement of the above references herein is not to be inferred as meaning that these are in any way relevant to the patentability of the presently disclosed subject matter.

### BACKGROUND

To date, noninvasive measurement of a person's hemodynamic parameters, such as blood pressure, has presented significant technical challenges. Monitoring vital parameters of a subject continuously during his/her daily routine can be very advantageous for identifying abnormal conditions at an early stage and such that the subject may be referred to receive essential medical care. Thus, the need of accurate and convenient to use noninvasive measurement devices are of need.

### GENERAL DESCRIPTION

The present disclosure discloses a wearable device for measuring physiological parameters of a subject while being worn. The device includes one or more sensors that perform the measurement of the physiological parameters. The sensors can be selected from either a light-based sensor or displacement sensor.

The light-based sensor includes at least one light source and at least one detector, defining together a sensing couple. The light source is configured to direct illumination towards a skin portion of the subject and the detector is configured to detect reflection of said illumination from the skin or tissues, such as arterioles or arteries below the skin, namely a PPG sensor. The detection of the reflected illumination is indicative of physiological parameters of the subject.

The displacement sensor is configured to sense a displacement of fine movements of the skin of the subject.

The device includes a skin contact member that has a skin contact surface facing an external side of the device that faces the skin of the subject. During measurements of one of the sensors, the contact surface engages the skin of the subject to allow performing measurements by at least one sensor.

The light-based sensor is configured to perform the measurement via the skin contact surface, namely the illumination of the light source propagates through the skin contact surface, interacts with the skin and tissues of the subject, and reflects therefrom to propagate via the skin contact surface towards the light detector.

The displacement sensor is configured to sense the displacement of the skin contact surface, which occurs due to small movements of the skin of the subject in response to expansion and contraction of blood vessels under the skin during blood cycles.

For allowing accurate performance of the measurements, the skin contact surface is required to displace smoothly in response to contact or pressure by the skin of the subject thereon. Furthermore, the design of the skin contact surface should allow functional engagement with the skin of the subject. Thus, the skin contact member is integrally formed with a first end of a flexible member allowing the smooth displacement thereof. A housing of the device is integral with a second end of the flexible member such that the skin contact member displaces along at least one axis with respect to the static housing. The integration of the three elements, the skin contact member, the flexible member, and the housing forms a continuous structure, which is advantageous and allows to obtain the desired accuracy of the fine measurements. The integration of the three parts is typically performed by one or more overmolding processes.

An aspect of the present disclosure provides a device for measuring physiological parameters of a subject. The device includes a housing that can be formed by one or more parts. The device further includes a skin contact member that one of its faces serves as a skin contact surface for contacting the skin of the subject. A flexible member laterally surrounding the skin contact member and is integral with the skin contact member and the housing. The flexible member is configured to permit the skin contact member to axially displace along an axis that is generally normal to the skin contact surface. The device further includes at least one sensor selected from: (i) a displacement sensor associated with the contact member for measuring the member's axial displacement; and (ii) a light sensor assembly having a light emitter and light detector couple, or more than one of each, for illuminating a skin portion via the skin contact surface and measuring a response to the illumination, i.e. a reflection from the skin or tissues below the skin surface.

It is to be noted that any combination of the described embodiments with respect to any aspect of this present disclosure is applicable. In other words, any aspect of the present disclosure can be defined by any combination of the described embodiments.

In some embodiments of the device, at least a portion of the following elements, the housing, the skin contact member and the flexible member are integrally formed to one another by one or more over-molding processes. An overmolding is a process where a single part is created using two or more different materials in combination. Typically, the first material, sometimes referred to as the substrate, is partially or fully covered by subsequent materials (overmold materials) during the manufacturing process. Typically, the overmolding processes are carried sequentially, a first process between two of the elements, and a second overmolding process between the resulted integration of the first two elements and the third element. These overmolding processes yield a smooth transition between the housing and the flexible member and between the flexible member and the skin contact member, namely an integration portion of the flexible member is flush with the skin contact surface. It may also form, in some embodiments, a waterproof casing, which makes the device to be waterproof that is obtained by the integration of the elements one to another.

In some embodiments of the device, the housing and the skin contact member are substantially made of a first material and the flexible member is substantially made of a second material. The term “substantially” defines that the majority, i.e. above 50% of the amount of material, either volume and/or weight, of the element is made of the same material. The element may include other materials, which are of a lower amount or volume than the substantial material.

In some embodiments of the device, the flexible member has a generally planar portion and a slanted portion. The planar portion extends between the housing and the slanted portion, and the slanted portion extending between the planar portion and the periphery of the skin contact surface of the skin contact member. The slanted portion actually extends between a first level to a second level, wherein the second level is elevated, at least at a non-biased state of the skin contact member, towards the external side of the device, with respect to the first level.

In some embodiments of the device, the planar portion is substantially parallel to the skin contact surface, namely not geometric but seen so to the unaided eye.

In some embodiments of the device, the flexible member is integral with the skin contact member such that the flexible member is flush with the skin contact surface.

In some embodiments of the device, the skin contact surface defines a plane that generally lies on the second level, namely the contact surface plane is elevated towards the external side with respect to the plane defined by the lateral portion of the flexible member and/or the plane defined by the bottom surface of the housing.

In some embodiments of the device, the skin contact surface includes one or more ECG electrodes. The one or more ECG electrodes are fixed on or integrated with the skin contact surface.

In some embodiments of the device, the skin contact surface comprises one or more light-transmissive, i.e. transparent, or translucent elements to permit transmission of light from the emitter and reflected light from the skin to the detector.

In some embodiments, the device includes at least one first light-transmissive element and at least one second light-transmissive element separated from the first light transmissive portion. The first light-transmissive element serves for allowing transmission of light therethrough from the at least one emitter that illuminates from a location below the skin contact surface towards the skin of the subject. The second light-transmissive element serves for allowing reflected light from the skin to reach the detector that is located below the skin contact surface.

In some embodiments, the device includes two or more light emitters, and each light emitter is associated with a respective first light-transmissive element. Each light-transmissive element is spatially separated from any other light-transmissive element to avoid undesired interference of light originated from two different light emitters.

In some embodiments of the device, at least two light emitters are configured to emit light of two different wavelengths ranges.

In some embodiments of the device, each light detector is associated with a respective second light-transmissive element, spatially separated from any other light-transmissive element.

In some embodiments of the device, each of the first light-transmissive elements abuts from the skin contact surface, namely at least a portion thereof abuts off any other portions of the skin contact surface. This configuration ensures that when a skin of a subject engages the contact surface, at least a portion of the skin contacts the first light-transmissive element and light emitted from the light emitters is guided towards the skin resulting in a high signal-to-noise ratio.

In some embodiments of the device, each of the first light-transmissive elements is dome-shaped and abuts off the skin contact surface, namely the dome-shaped portion abuts off other portions of the skin contact surface.

In some embodiments of the device, the second light transmissive element is planar, e.g. substantially flush with the contact surface.

In some embodiments of the device, the first light transmissive element is being a part of an optical arrangement for guiding light emitted from a respective light emitter, e.g. a respective LED. dome that functions as a focusing lens. Namely, the first light transmissive element is shaped as a focusing lens for the light emitted from a respective light source towards the skin.

In some embodiments of the device, the skin contact surface is formed with a central depression that accommodates the one or more light transmissive elements.

In some embodiments of the device, the flexible member and the skin contact member are concentric.

In some embodiments of the device, the flexible member has an integration interface portion engaging the skin contact member. The integration interface portion, which constitutes the inner end of the flexible member, is thicker than other portions of the flexible member.

In some embodiments of the device, the flexible member and the skin contact member are integral to one another via an integration interface having a longitudinal cross-section profile designed for increasing the surface area of the integration interface to thereby increasing the integration strength.

In some embodiments of the device, the longitudinal cross-section profile is designed such that the surface area of the integration surface is greater than a minimal surface area of the integration interface. The minimal surface area is defined by a straight vertical longitudinal cross-section profile being normal to a plane defined by the contact surface.

In some embodiments of the device, the surface of the integration interface is non-planar.

In some embodiments of the device, the flexible member is integral to the skin contact member via an inner portion thereof. The inner portion is formed of a first inner segment and a second inner segment, and a gap is spanned between the first and the second segments to define an inner depression. The skin contact member comprises an external portion corresponding to said inner portion to allow said integration. Namely, the outer portion of the skin contact member is formed of a first external segment being integral with the first inner segment, a second external segment being integral with the second inner segment and a third external segment that fits into said depression that is formed between the first and the second inner segments of the flexible member.

In some embodiments of the device, the first and second inner segments laterally extending from the slanted portion. In other words, the first and second inner segments protrude from the slanted portion and define planes parallel to the plane defined by the contact surface.

In some embodiments of the device, the first inner segment laterally extending to an extent greater than the second inner segment.

In some embodiments of the device, the flexible member has a segment extending between a peripheral section, which is configured for coupling with the housing, and an inner section configured for tight coupling with a counterpart external portion of the contact member.

In some embodiments of the device, the inner section has an undulated surface for engaging a corresponding surface of said external portion.

In some embodiments of the device, the inner section is thicker than the segment.

In some embodiments of the device, the inner section has two engaging portions sandwiching said external portion.

Yet another aspect of the present disclosure provides a method for manufacturing a device for measuring physiological parameters of a subject. The method comprising (i) molding a first part being the housing of the device and a second part being the contact member of the device, the skin contact member defining a skin contact surface at one of its faces for contacting the skin of the subject; (ii) molding a flexible member to integrate with the housing at its outer end and integrate with the skin contact member at its inner end such that the flexible member laterally surrounding the skin contact member, and permitting the skin contact member to axially displace along an axis normal to the skin contact surface. The device is further characterized with at least one of: (1) a displacement sensor associated and movable with the contact member for measuring the member's axial displacement and/or (2) a light sensor assembly having a light emitter and light detector couple for illuminating a skin portion via the skin contact surface and measuring a response to the illumination.

In some embodiments of the method, the molding of the flexible member is carried out by an over-molding process.

In some embodiments of the method, the housing and the skin contact member are made of a first material and the flexible member is made of a second material.

In some embodiments, the method further comprises injecting light-transmissive material to intended portions at the skin contact member to form at least one first light transmissive element and at least one second light transmissive element separated from the first light transmissive portion. Said first light-transmissive element is intended for allowing transmission of light therethrough from said at least one emitter towards the skin of the subject and said second light-transmissive element is intended for allowing reflected light from the skin to reach the detector.

In some embodiments of the method, each of the first and second light-transmissive elements is separated from any other light-transmissive element. Each first light-transmissive element is associated with a respective single light emitter and each second light-transmissive element is associated with a respective single light detector.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1B are schematic illustrations of non-limiting examples of different parts of the device according to an embodiment of the present disclosure. FIG. 1A is a bottom view of the device; FIG. 1B is a longitudinal cross-section of the skin contact member.

FIGS. 2A-2G are illustrations of different views of a non-limiting example of the device according to an embodiment of the present disclosure. FIG. 2A is a bottom view;

FIG. 2B is a perspective view of the bottom part of the device; FIG. 2C is a side-view of the bottom part of the device; FIG. 2D is a longitudinal cross-section of the bottom part of the device; FIG. 2E is a perspective view of the top part of the device; FIG. 2F is a longitudinal cross-section view of the device showing the flexible member and the skin contact member and the integration thereof; FIG. 2G is a longitudinal cross-section focusing on the integration interface between the flexible member and the skin contact member.

FIGS. 3A and 3B are perspective views of the bottom part and top part of the device, respectively.

### DETAILED DESCRIPTION OF EMBODIMENTS

The following figures are provided to exemplify embodiments and realization of the invention of the present disclosure.

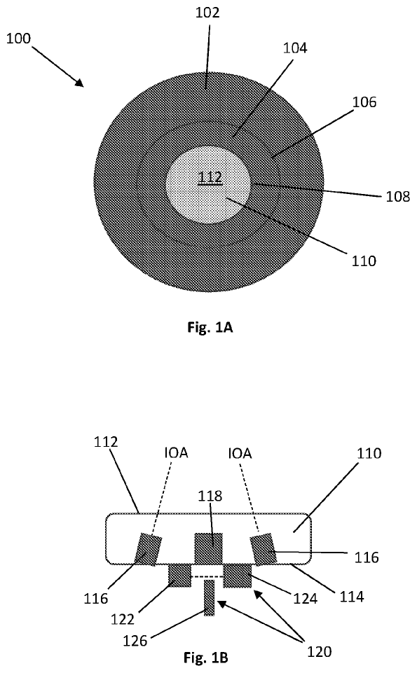

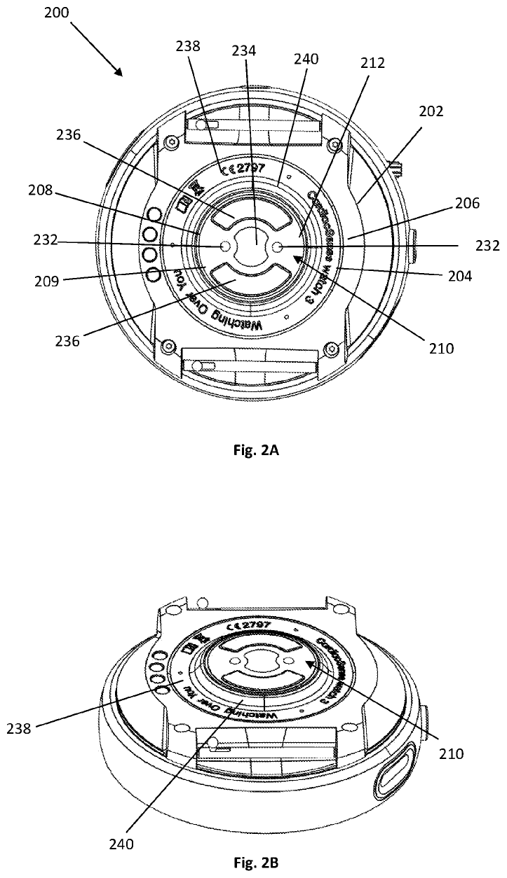

Reference is first made to FIG. 1A, which is a schematic illustration of a bottom view of a non-limiting example of a portion of measuring device according to an embodiment of the present disclosure. FIG. 1A shows a bottom part of a housing 102 of the device 100. The entire housing 102 may be constituted by one or more parts and FIG. 1A shows only a part thereof. A flexible member 104 laterally extends between a peripheral section 106 that is integral with the housing 102 and an inner section 108 that is integral with a skin contact member 110. The housing 102 laterally surrounding the flexible member 104 and the flexible member laterally surrounding the skin contact member 110. An external face of the skin contact member 110 defines a skin contact surface 112 for contacting the skin of a subject. The flexible member 104 is configured to allow the displacement of the skin contact member 110 at least along one axis normal to a plane defined by the skin contact surface 112. The skin contact member 110 is further configured to hold at least one sensor in a fixed association with the contact surface. The sensor is either (i) a displacement sensor for measuring a displacement of the contact surface, e.g. an optical-based sensor such as exemplified in PCT publication No. WO 2019/215723; (ii) a light-based sensor for measuring physiological parameters by illuminating light towards the skin of the subject, via the skin contact surface, and sensing the reflections therefrom, or from tissues below the skin, e.g. a PPG sensor; (iii) a combination of displacement sensor and light-based sensor. Reference is now made to FIG. 1B, which is a schematic illustration of a longitudinal cross-section of an example of the contact skin member showing optional configuration of integration of the displacement sensor and the light-based sensor to the skin contact member. The light-based sensor components are fixed to internal surface 114 of the skin contact member 110, these components include one or more light sources 116 and one or more light detectors 118. The light sources 116 are configured to illuminate the skin of the subject through the contact surface 112 along an illumination optical axis IOA and the light reflected from the skin is transmitted through a portion of the skin contact surface 112 and detected by the light detector 118. The detection of the reflected light is indicative of physiological parameters of the subject, e.g. heart rate, blood pressure or respiration rate. Furthermore, a displacement sensor 120 may be fixed to the internal surface 114 or to another internal part of the skin contact member to measure the displacement degree of the skin contact surface 112. For example, the displacement sensor may be formed by a light source 122 that is configured to emit light L towards a light detector 124 and a blocking element 126 is designed to block a portion of the emitted light in proportion to the displacement of the skin contact surface 112. It is to be noted that the skin contact member may be fixed with only one sensor, e.g. only with the displacement sensor and the design of the sensors may be different that is described with respect to FIG. 1B. The sensors shown in FIG. 1B are mere examples of one realization of embodying sensors in the skin contact member.

Referring to FIG. 1A, it is noted that the housing 102, the flexible member 104 and the skin contact member 110 are integrally formed to one another by one or more overmolding processes. By this integration, the structure that is defined by the three elements is continuous. This also provides a certain degree of water resistance for avoiding liquid penetration into the enclosure of the device.

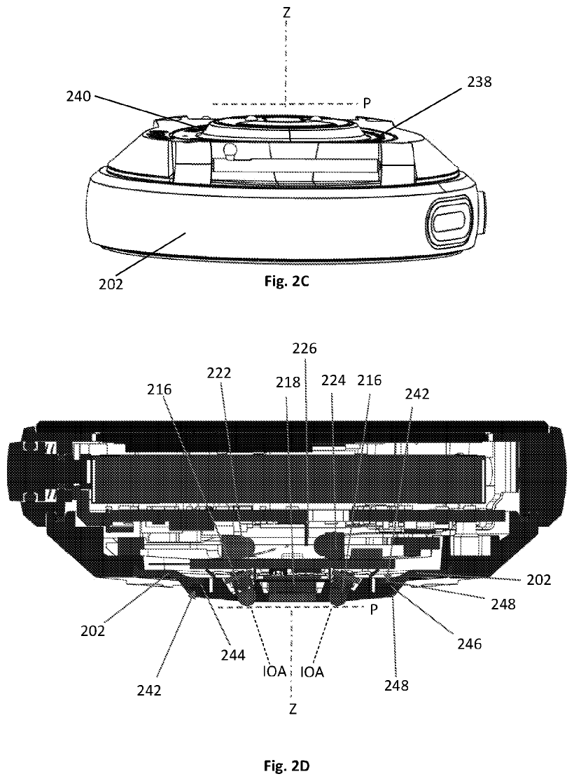

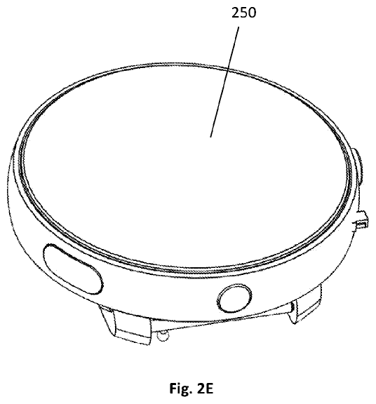

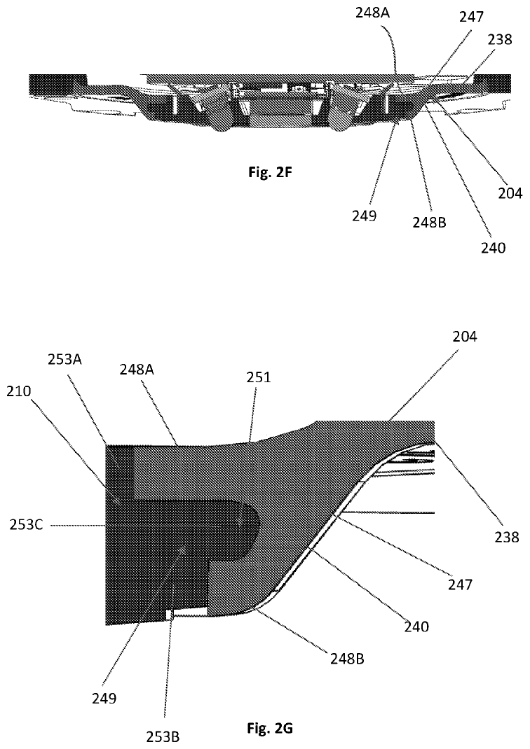

In the figures throughout the application, like elements of different figures were given similar reference numerals shifted by the number of hundreds corresponding to the number of the respective figure. For example, element 204 in FIGS. 2A-2E serves the same function as element 104 in FIGS. 1A-1B.

Reference is now made to FIGS. 2A-2E, which are different views of schematic illustration of a non-limiting example of a device according to an embodiment of the present disclosure. FIG. 2A shows a device 200 that includes a housing 202, which its bottom part shows. The housing 202 laterally surrounds a flexible member 204 and integral with a peripheral section 206 thereof. An inner section 208 of the flexible member 204 surrounds and integral with a peripheral portion 209 of a skin contact member 210. The skin contact member has a skin contact surface 212 facing to an external side of the device 200. The skin contact surface 212 includes first and second light-transmissive portions 232 and 234, respectively. The first light-transmissive portions 232 serve for transmission of light from the light sources 216 (as can be seen in FIG. 2D) below the skin contact surface 212 towards the skin of the subject and the second light-transmissive portion serves for allowing transmission of light reflection of the light sources to be received in a detector 218 (as can be seen in FIG. 2D) below the skin contact surface 212. Each of the light-transmissive portions 232, 234 is laterally separated from any other light-transmissive portion 232, 234. Namely, any light-transmissive portion is formed on a different portion of the contact surface 212, isolated from any other light-transmissive portion. The skin contact surface accommodates the first and second light-transmissive portions 232 and 234. The first light-transmissive portions 232 are disposed on opposite sides of the second light-transmissive portion 234. The illumination optical axis of any of the light source associated with the respective first light-transmissive portions 232 may be either parallel to the displacement axis Z or can form an angle with the axis, e.g. an acute angle, such that the light emitted from the light source is directed towards the direction of the light second light-transmissive portion 234 and the light detector 218. The first light-transmissive portions 232 include portions that are dome-shaped protruding above the plane defined by all other elements of the contact surface 212, namely the first light-transmissive portions 232 protrude above any other portion of the skin contact surface 212, including the second light-transmissive portion 234 and the ECG electrodes 236 that are formed thereon. As mentioned, the skin contact surface 212 further includes two ECG electrodes 236 formed on two different portions thereof. The flexibility of the flexible member 204 allowing the skin contact member 210 to displace along at least one axis Z, as can be best seen in FIGS. 2C-2D. The axis Z is normal to a plane P defined by the skin contact surface 212. It is to be noted that the skin contact surface is generally planar, though it can deviate from an exact geometrical plane, to be adapted for the contour of the wrist of the subject. The extent of the displacement of the skin contact member is measured by an optically-based displacement sensor 220 that is formed of a light emitter 222 that is configured to directly emit light towards a light detector 224, and a light-blocking element 226 is placed therebetween to block an amount of light that reaches the light detector 224 proportionally to the displacement of the skin contact member 210.

The flexible member 204 is constituted by two integral and continuous portions, a planar portion 238 and a slanted portion 240. The planar portion 238, which is more peripheral, extends between the housing 202 and the slanted portion 240 and generally spans a common plane with the bottom part of the housing 202 it is linked and continuous thereto. The slanted portion 240 extends between the inner section of the planar portion 238 and the periphery 209 of the skin contact surface 212 of the skin contact member 210. Furthermore, the slanted portion 240 extends between a first plane spanned by the planer portion 238 and a second plane spanned by the skin contact surface 212 such that the skin contact member spans a plane that is elevated with respect to the plane of the housing 202 and the planar portion 238. This provides a better engagement between the skin contact surface 212 and the skin portion of the subject.

The flexible member 204 and the skin contact member 210 are concentric, the first being ring-shaped and the latter is circular.

FIG. 2D is a longitudinal cross-section of the device showing some of the interior of the bottom part of the device and outlining the cross-section front part. The outline in FIG. 2D shows the integration interface 242 between the flexible member 204 and the skin contact member 210. The integration interface is designed so as to obtain a relatively large integration surface area for increasing the integration strength. In this example, the integration interface 242 defines a longitudinal cross-section profile 244 that is patterned to be greater than a straight vertical line between a top end of the integration interface and a bottom part thereof, which is normal to the plane of the skin contact surface 212. The longitudinal cross-section profile 244 has a generally undulated profile that yields a structure in which an external segment 246 of the skin contact member 210 is sandwiched between two segments 248 of the flexible member 204. Reference is specifically made to FIGS. 2F and 2G, which are cross-sectional views of the device showing the integration interface, wherein FIG. 2G is a zoom in view of the integration interface. The integration interface is constituted by the integration of an inner portion 247 of the flexible member 204 and external portion 249 of the skin contact member 210. The inner portion 247 of the flexible member 204 is formed of a first inner segment 248A and a second inner segment 248B. Each The first and second inner segments 248A and 248B protrude from different portions of the slanted portion 240 of the flexible member 204. The first inner segment 248A protrude from a portion more proximal to the planar portion 238 of the flexible member 204 and the second inner segment 248B protrude from a portion more proximal to the skin contact surface 212 of the skin contact member 210. A depression 251 is formed between the first and the second inner segments 248A and 248B. The external portion 249 of the skin contact member 210 has a contour that is corresponding to the inner portion 247 of the flexible member 204. Thus, the inner portion 247 of the flexible member 204 comprises is formed of a first external segment 253A being integral with the first inner segment 248A, a second external segment 253B being integral with the second inner segment 248B, and a third external segment 253C that fits in and being integral with the depression 251. The first and the second inner segments 248A and 248B lie on parallel planes, which are parallel to the plane defined by the planar portion 238 of the flexible member 204. The first inner segment 248A laterally extending to a greater extent than the second inner segment 248B. Furthermore, FIG. 2D exemplifies that the two light sources 216, e.g. LEDs, are fixed to the contact member 212 and tilted to form an angle between their illumination optical axes IOA and the displacement axis Z. The first light-transmissive portions 232 that are associated with the light sources 216 are dome-shaped and protruding over other portions of the skin contact surface 212 that define its plane.

FIG. 2E shows the top part of the measuring device 200 and its housing 202. The measuring device typically includes a display 250 for displaying the measured data of the physiological parameters of the subject.

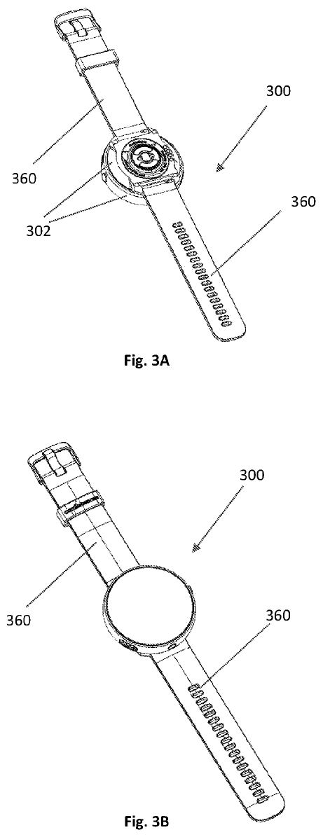

FIGS. 3A-3B are schematic illustrations of a non-limiting example of different views of the device of FIGS. 2A-2E being in the form of a wristwatch. The housing 302 of the device 300 is linked to wristbands 360 for fastening the wristwatch to a wrist of the subject for allowing continuous measurement of his/her physiological parameters by measuring either movement of the skin or illumination response, i.e. reflection of light illuminated from a light source of the device towards the skin or tissues below the skin.

1. A device for measuring physiological parameters of a subject, comprising:
a housing;
a skin contact member defining a skin contact surface at one of its faces for contacting the skin of the subject;
a flexible member
laterally surrounding the skin contact member,
a first end of the flexible member is being integral with the skin contact member and a second end is being integral with the housing, and
permitting the skin contact member to axially displace along an axis normal to the skin contact surface with respect to the static housing; and
(i) a displacement sensor associated with the contact member for measuring the skin contact member's axial displacement along said axis and (ii) a light sensor assembly having a light emitter and light detector couple for illuminating a skin portion via the skin contact surface and measuring a response to the illumination;
wherein the skin contact surface comprises at least one first light transmissive element and at least one second light transmissive element separated from the first light transmissive element, said first light-transmissive element is intended for allowing transmission of light therethrough from said light emitter towards the skin of the subject and said second light-transmissive element is intended for allowing reflected light from the skin to reach the detector;
and
wherein a portion of said at least one first light transmissive element protrudes above any other portions of said skin contact surface.

2. The device of claim 1, at least portion of the housing, the skin contact member and the flexible member are integrally formed by one or more over-molding processes.

3. The device of claim 1, wherein the flexible member has a generally planar portion and a slanted portion, the planar portion extending between the housing and the slanted portion and the slanted portion extending between the planar portion and the periphery of the skin contact surface of the skin contact member.

4. (canceled)

5. The device of claim 1, wherein the skin contact surface comprises one or more ECG electrodes.

6. The device of claim 1, wherein each of the first and second light-transmissive elements is separated from any other light-transmissive element, each first light-transmissive element is associated with a respective single light emitter and each second light-transmissive element is associated with a respective single light detector.

7. The device of claim 6, wherein the at least one first light transmissive element serves as a guiding optics for guiding light emitted from a respective light emitter associated with said first light transmissive element.

8. The device of claim 1, wherein the first light transmissive element is dome-shaped.

9. The device of claim 1, wherein the second light transmissive element is planar.

10. The device of claim 8, wherein said first light transmissive element is shaped and functions as a focusing lens.

11. The device of claim 1, wherein the flexible member has an integration interface portion engaging the skin contact member, said interface being thicker than other portions of the flexible member.

12. The device of claim 1, wherein the flexible member and the skin contact member are integral to one another via an integration interface having a longitudinal cross-section profile designed for increasing the surface area of the integration interface.

13. The device of claim 12, wherein the longitudinal cross-section profile is designed such that the surface area of the integration surface is greater than a minimal surface area of the integration interface defined by a straight vertical longitudinal cross-section profile extending normal to a plane defined by the contact surface; and wherein the surface of the integration interface is non-planar.

14. (canceled)

15. The device of claim 1, wherein the flexible member comprises an inner portion and the skin contact member comprises an external portion such that the inner portion is integral with the external portion, said inner portion is formed of a first inner segment and a second inner segment defining a depression therebetween;
said external portion is formed of a first external segment being integral with the first inner segment, a second external segment being integral with the second inner segment and a third external segment that fits into and integral with said depression.

16. The device of claim 15, wherein the flexible member has a planar portion and a slanted portion, the planar portion extending between the housing and the slanted portion and the slanted portion extending between the planar portion and the periphery of the skin contact surface of the skin contact member; and wherein the first and second inner segments protruding from said slanted portion and spanning parallel planes that are parallel to the planar portion.

17. The device of claim 16, wherein the first inner segment protruding to an extent greater than the second inner segment.

18. The device of claim 1, wherein the flexible member has a segment extending from a peripheral section configured for coupling with the housing and an inner section configured for tight coupling with a counterpart external portion of the contact member.

19. The device of claim 18, wherein said inner section has two engaging portions sandwiching said external portion.

20. A method for manufacturing a device for measuring physiological parameters of a subject, comprising:
(i) molding a first part being the housing of the device and a second part being the contact member of the device, the skin contact member defining a skin contact surface at one of its faces for contacting the skin of the subject;
(ii) molding a flexible member to integrate with the housing at its outer end and integrate with the skin contact member at its inner end such that the flexible member laterally surrounding the skin contact member, and permitting the skin contact member to axially displace along an axis normal to the skin contact surface;
(iii) injecting light-transmissive material to intended portions at the skin contact member to form at least one first light transmissive element and at least one second light transmissive element separated from the first light transmissive portion, said first light-transmissive element is intended for allowing transmission of light therethrough from a light emitter towards the skin of the subject and said second light-transmissive element is intended for allowing reflected light from the skin to reach the detector, wherein each of the first and second light-transmissive elements is separated from any other light-transmissive element, and each first light-transmissive element is associated with a respective single light emitter and each second light-transmissive element is associated with a respective single light detector;
wherein the device is further characterized with (1) a displacement sensor associated and movable with the contact member for measuring the member's axial displacement along said axis and (2) a light sensor assembly having said light emitter and light detector couple for illuminating a skin portion via the skin contact surface and measuring a response to the illumination; and
wherein a portion of said at least one first light transmissive element is domed-shaped and protrudes above any other portions of said skin contact surface;
wherein the skin contact surface comprises ECG electrodes.

21. The method of claim 20, wherein (ii) is carried out by an over-molding process; and wherein the housing and the skin contact member are made of a first material and the flexible member is made of a second material.

22. (canceled)

23. A device for measuring physiological parameters of a subject, comprising:
a housing;
a skin contact member defining a skin contact surface at one of its faces for contacting the skin of the subject;
a flexible member
laterally surrounding the skin contact member,
a first end of the flexible member is being integral with the skin contact member and as second end is being integral with the housing, and
permitting the skin contact member to axially displace along an axis normal to the skin contact surface with respect to the static housing; and
a (i) a displacement sensor associated with the contact member for measuring the skin contact member's axial displacement along said axis and (ii) a light sensor assembly having a light emitter and light detector couple for illuminating a skin portion via the skin contact surface and measuring a response to the illumination;
wherein the skin contact surface comprises at least one first light transmissive element and at least one second light transmissive element separated from the first light transmissive element, said first light-transmissive element is intended for allowing transmission of light therethrough from said light emitter towards the skin of the subject and said second light-transmissive element is intended for allowing reflected light from the skin to reach the detector; and
wherein a portion of said at least one first light transmissive element is dome-shaped and protrudes above any other portions of said skin contact surface;
wherein the skin contact surface comprises ECG electrodes.
